(19) United States
(12) Patent Application Publication
(10) Pub. No.: US 2026/0345169 A1
(43) Pub. Date: Nov. 5, 2026

(54) DELIVERY DEVICES FOR TREATMENT OF VASCULAR DEFECTS

(71) Applicant: MICROVENTION, INC., Aliso Viejo, CA (US)

(72) Inventors: Todd Hewitt, Laguna Niguel, CA (US); Parker Milhous, Santa Ana, CA (US); Leonardo Villalba, La Habra, CA (US)

(21) Appl. No.: 19/101,980

(22) PCT Filed: Aug. 3, 2023
(86) PCT No.: PCT/US2023/029373

§ 371 Date: Feb. 7, 2025

Related U.S. Application Data
(60) Provisional application No. 63/396,410, filed on Aug. 9, 2022.

(51) Int. Cl. A61B 17/12; A61B 17/00

(52) U.S. Cl. CPC A61B 17/12113; A61B 17/1215; A61B 17/12172; A61B 2017/00867; A61B 2017/1205

(57) ABSTRACT

Delivery systems and methods of delivery are described that include a brake to prevent an occlusive device from jumping forward during deployment. The delivery system may include a self-expandable implant having radially constrained state within a lumen of a catheter and an expanded state after deployment; and a pusher comprising a distal end, a distal region, and a brake located in the distal region of the pusher for frictionally engaging an inner wall of the catheter during deployment of the self-expandable implant, and wherein the implant is releasably coupled to the distal end of the pusher. In some embodiments, the brake may be at least one leaf spring, a braid, or a section of increased diameter on the pusher. In some embodiments, the brake may be made from a shape-memory material and have first and second configurations.

### CROSS-REFERENCE TO RELATED APPLICATIONS

This application is a national stage patent application under 35 U.S.C. § 371 of International Application No. PCT/US23/29373, filed Aug. 3, 2023, which claims the benefit of U.S. Provisional Application Ser. No. 63/396,410, filed Aug. 9, 2022, both of which are hereby expressly incorporated by reference in their entireties for all purposes.

### STATEMENT REGARDING FEDERALLY SPONSORED RESEARCH OR DEVELOPMENT

Not applicable.

### FIELD OF THE INVENTION

Embodiments of devices and methods herein are directed to improved delivery systems for delivering implants.

### BACKGROUND OF THE INVENTION

The mammalian circulatory system is comprised of a heart, which acts as a pump, and a system of blood vessels which transport the blood to various points in the body. Due to the force exerted by the flowing blood on the blood vessel the blood vessels may develop a variety of vascular defects. One common vascular defect known as an aneurysm results from the abnormal widening of the blood vessel. Typically, vascular aneurysms are formed as a result of the weakening of the wall of a blood vessel and subsequent ballooning and expansion of the vessel wall. If, for example, an aneurysm is present within an artery of the brain, and the aneurysm should burst with resulting cranial hemorrhaging, death could occur.

Surgical techniques for the treatment of cerebral aneurysms typically involve a craniotomy requiring creation of an opening in the skull of the patient through which the surgeon can insert instruments to operate directly on the patient's brain. For some surgical approaches, the brain must be retracted to expose the parent blood vessel from which the aneurysm arises.

Once access to the aneurysm is gained, the surgeon places a clip across the neck of the aneurysm thereby preventing arterial blood from entering the aneurysm. Upon correct placement of the clip the aneurysm will be obliterated in a matter of minutes. Surgical techniques may be effective treatment for many aneurysms. Unfortunately, surgical techniques for treating these types of conditions include major invasive surgical procedures which often require extended periods of time under anesthesia involving high risk to the patient. Such procedures thus require that the patient be in generally good physical condition in order to be a candidate for such procedures.

Various alternative and less invasive procedures have been used to treat cerebral aneurysms without resorting to major surgery. One approach to treating aneurysms without the need for invasive surgery involves the placement of sleeves or stents into the vessel and across the region where the aneurysm occurs. Such flow diverter devices maintain blood flow through the vessel while reducing blood pressure applied to the interior of the aneurysm. Certain types of stents are expanded to the proper size by inflating a balloon catheter, referred to as balloon expandable stents, while other stents are designed to elastically expand in a self-expanding manner. Some stents are covered typically with a sleeve of polymeric material called a graft to form a stent-graft. Stents and stent-grafts are generally delivered to a preselected position adjacent a vascular defect through a delivery catheter. In the treatment of cerebral aneurysms, covered stents or stent-grafts have seen very limited use due to the likelihood of inadvertent occlusion of small perforator vessels that may be near the vascular defect being treated.

In addition, current uncovered stents are generally not sufficient as a stand-alone treatment. In order for stents to fit through the microcatheters used in small cerebral blood vessels, their density is usually reduced such that when expanded there is only a small amount of stent structure bridging the aneurysm neck. Thus, they do not block enough flow to cause clotting of the blood in the aneurysm and are thus generally used in combination with vaso-occlusive devices, such as the coils discussed above, to achieve aneurysm occlusion.

Some procedures involve the delivery of embolic or filling materials into an aneurysm. The delivery of such vaso-occlusion devices or materials may be used to promote hemostasis or fill an aneurysm cavity entirely. Vaso-occlusion devices may be placed within the vasculature of the human body, typically via a catheter, either to block the flow of blood through a vessel with an aneurysm through the formation of an embolus or to form such an embolus within an aneurysm stemming from the vessel. A variety of implantable, coil-type vaso-occlusion devices are known. The coils of such devices may themselves be formed into a secondary coil shape, or any of a variety of more complex secondary shapes. Vaso-occlusive coils are commonly used to treat cerebral aneurysms but suffer from several limitations including poor packing density, compaction due to hydrodynamic pressure from blood flow, poor stability in wide-necked aneurysms, and complexity and difficulty in the deployment thereof as most aneurysm treatments with this approach require the deployment of multiple coils. Coiling is less effective at treating certain physiological conditions, such as wide neck cavities (e.g., wide neck aneurysms) because there is a greater risk of the coils migrating out of the treatment site.

A number of aneurysm neck bridging devices with defect spanning portions or regions have been attempted, however, none of these devices have had a significant measure of clinical success or usage. A major limitation in their adoption and clinical usefulness is the inability to position the defect spanning portion to assure coverage of the neck. Existing stent delivery systems that are neurovascular compatible (i.e., deliverable through a microcatheter and highly flexible) do not have the necessary rotational positioning capability. Another limitation of many aneurysm bridging devices described in the prior art is the poor flexibility. Cerebral blood vessels are tortuous, and a high degree of flexibility is required for effective delivery to most aneurysm locations in the brain.

What has been needed are devices and methods for delivery and use in small and tortuous blood vessels that can substantially block the flow of blood into an aneurysm, such as a cerebral aneurysm, with a decreased risk of inadvertent aneurysm rupture or blood vessel wall damage. In addition, what has been needed are methods and devices suitable for blocking blood flow in cerebral aneurysms over an extended period of time without a significant risk of deformation, compaction, or dislocation.

Intrasaccular occlusive devices are part of a newer type of occlusion device used to treat various intravascular conditions including aneurysms. They are often more effective at treating these wide neck conditions, or larger treatment areas. The intrasaccular devices comprise a structure that sits within the aneurysm and provides an occlusive effect at the neck of the aneurysm to help limit blood flow into the aneurysm. The rest of the device comprises a relatively conformable structure that sits within the aneurysm helping to occlude all or a portion of the aneurysm. Intrasaccular devices typically conform to the shape of the treatment site. These devices also occlude the cross section of the neck of the treatment site/aneurysm, thereby promoting clotting and causing thrombosis and closing of the aneurysm over time. In larger aneurysms, there is a risk of compaction where the intrasaccular device can migrate into the aneurysm and leave the neck region.

For any sized aneurysm, there may be numerous different types of sizes of occlusive devices that could be chosen by the physician to treat the aneurysm, where the devices may differ in height and diameter. The implants may also have different expanded shapes, e.g., barrel or spherical shape. Thus, many different sized and models of implants may have approximately the same volume as the aneurysm to be treated, and therefore are an acceptable “volume match” for the aneurysm.

As self-expanding implants are deployed in an aneurysm, the self-expanding implant may “jump” or move forward a distance out of the delivery catheter when a proximal end of the implant exits from the delivery catheter. Thus, there is a need for delivery devices that minimize “jumping” of the implant during delivery.

The following embodiments address this issue by utilizing a brake system incorporated in the delivery device.

### SUMMARY OF THE INVENTION

An implant delivery system is described that can be used to deliver an implant to treat a variety of conditions, including aneurysms and neurovascular aneurysms. In some embodiments, the occlusion device is configured as an intrasaccular device, and the delivery system includes a brake to prevent the occlusion device from jumping out of the catheter such that there is a space or distance between a proximal end of the implant and a distal end of the catheter upon deployment of occlusion device.

Delivery systems and methods of delivery are described that include a brake to prevent an occlusive device from jumping forward during deployment. The delivery system may include a self-expandable implant having radially constrained state within a lumen of a catheter and an expanded state after deployment; and a pusher comprising a distal end, a distal region, and a brake located in the distal region of the pusher for frictionally engaging an inner wall of the catheter during deployment of the self-expandable implant, and wherein the implant is releasably coupled to the distal end of the pusher. In some embodiments, the brake may be at least one leaf spring, a braid, or a section of increased diameter on the pusher. In some embodiments, the brake may be made from a shape-memory material and have first and second configurations.

### BRIEF DESCRIPTION OF THE DRAWINGS

These and other aspects, features, and advantages of which embodiments of the invention are capable of will be apparent and elucidated from the following description of embodiments of the present invention, reference being made to the accompanying drawings, in which:

The various figures included show the occlusive device according to one or more embodiments.

FIG. 1A is an elevation view of a device for treatment of a patient's vasculature.

FIG. 1B is a bottom perspective view of an embodiment of a device for treatment of a patient's vasculature.

FIGS. 2A-2C depict how an implant could jump forward during deployment from the catheter.

FIGS. 3A-3B show an exemplary embodiment of a delivery system with a brake.

FIGS. 4A-4C show an exemplary embodiment of a delivery system with a brake.

FIGS. 5A-5F show exemplary embodiments of brake systems with different implants

FIGS. 6A-6B show exemplary embodiments of a brake system with two configurations.

FIGS. 7A-7E show alternative embodiments of brake systems.

### DESCRIPTION OF EMBODIMENTS

The presented embodiments shall generally relate to delivery systems for occlusive devices.

### Occlusive Devices

Intrasaccular occlusive devices that include a permeable shell formed from a woven or braided mesh have been described in US 2012/0283768, US 2017/0095254, US 2016/0249934, US 2016/0367260, US 2016/0249937, and US 2018/0000489, all of which are hereby expressly incorporated by reference in their entirety for all purposes.

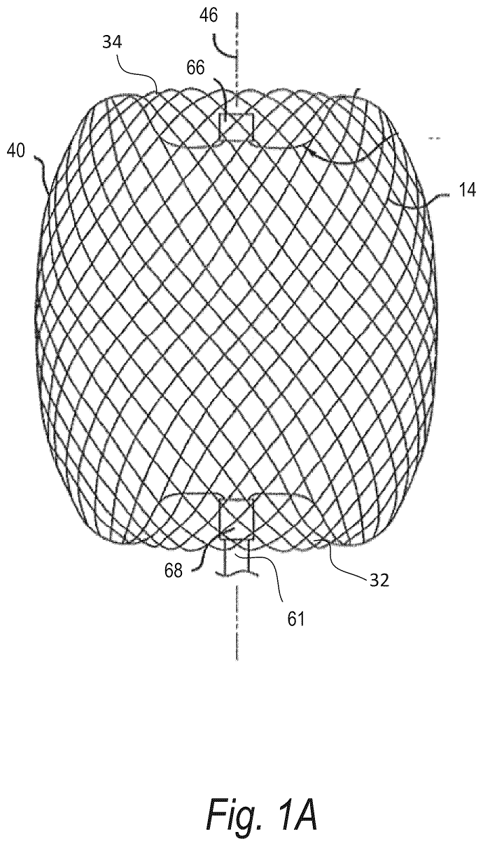

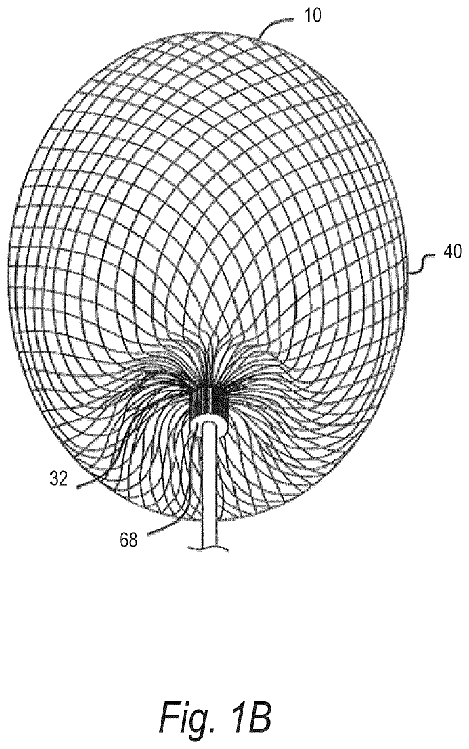

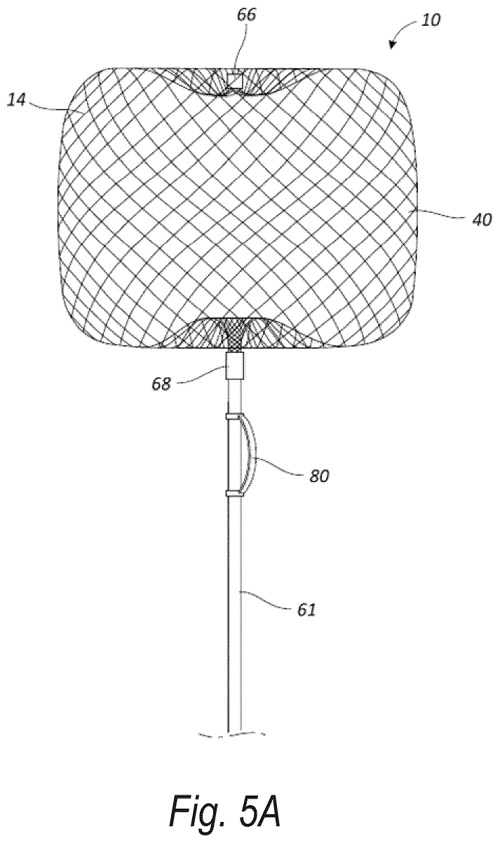

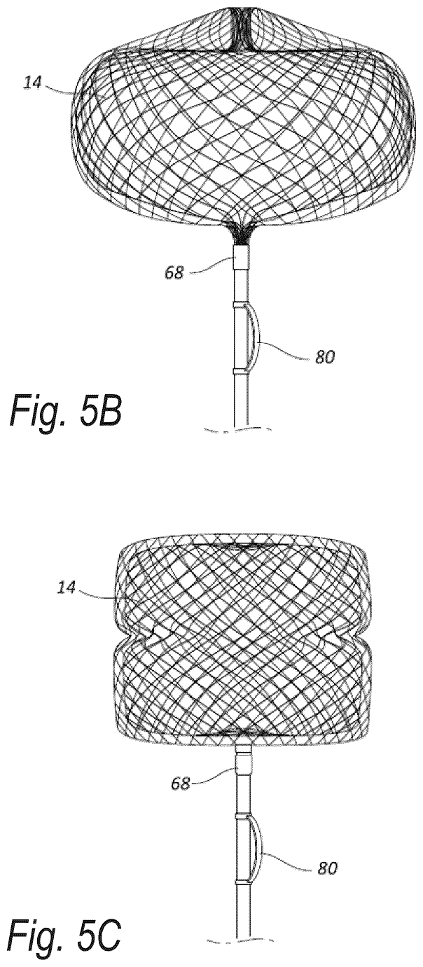

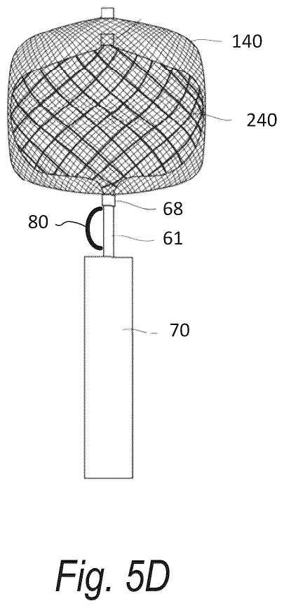

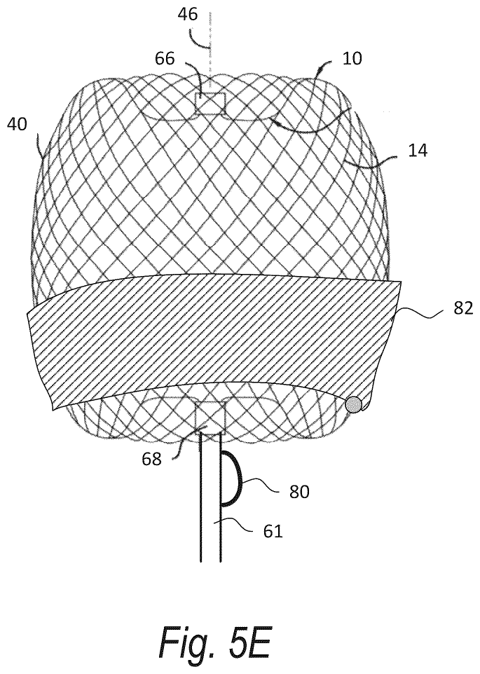

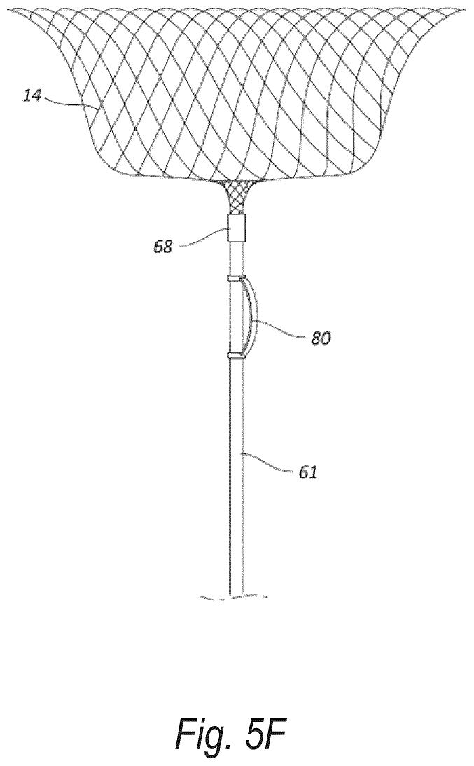

In some embodiments, as depicted in FIGS. 1A-1B, an intrasaccular device has a permeable shell 40 made from a woven mesh of filaments. The permeable shell 40 has a proximal end 32, a distal end 34, a longitudinal axis 46, and comprises a plurality of elongate resilient filaments 14. The filaments 14 may have a woven structure and may be secured relative to each other at the proximal ends and distal ends of the permeable shell 40, or alternatively, only at the proximal end (see, e.g., FIGS. 5B and 5F). The permeable shell 40 of the device 10 may have a radially constrained elongated state configured for delivery within a microcatheter, with the thin woven filaments extending longitudinally from the proximal end to the distal end of each permeable shell radially adjacent each other along a length of the filaments. The permeable shell 40 may have an expanded relaxed, or at rest, state with a longitudinally shortened configuration relative to the radially constrained state. In the expanded, at rest state, the woven filaments 14 form the self-expanding resilient permeable shell 40 in a smooth path radially expanded from a longitudinal axis of the permeable shell between the proximal end and distal end. The woven structure of the filaments forming the permeable shell includes a plurality of openings in the permeable shell 40 formed between the woven filaments. The braided mesh of the permeable shell 40 may also define an interior cavity.

The expanded state of the permeable shell 40 may have a diameter of about 4 mm, alternatively about 5 mm, alternatively about 6 mm, alternatively about 7 mm, alternatively about 8 mm, alternatively about 9 mm, alternatively about 10 mm, alternatively about 11 mm. The expanded state of the permeable shell 140 can have a height or length of about 2.6 mm, about 3 mm, about 3.6 mm, about 4 mm, about 4.6 mm, about 5 mm, about 5.6 mm, about 6 mm, about 6.6 mm, about 7 mm, about 7.6 mm, about 8 mm, about 8.6 mm, about 9 mm, about 9.6 mm, or about 10 mm.

The permeable shell 40 may be made from a plurality of wires or filaments 14 braided into a mesh, e.g., a tubular mesh. The permeable shell 140 may be made from about 96 to about 240 wires or filaments, alternatively about 96 to 180 wires or filaments, alternatively about 108 to about 180 wires or filaments, alternatively about 96 wires or filaments or more, alternatively about 108 wires or filaments or more, alternatively about 150 wires or filaments or more, alternatively about 180 wires or filaments or more. The diameter of the wires or filaments making up the exterior permeable shell 40 may be between about 0.0005″ to about 0.00075″, alternatively about 0.0004″ to about 0.0008″, alternatively about 0.0003″ to about 0.0009″, alternatively between about 0.00125″ to about 0.002″, alternatively about 0.001″ to about 0.002″, alternatively about 0.001″ to about 0.003″.

As described with respect to other embodiments of permeable shells, the filaments may be made from metals and metal alloys such as nitinol, platinum, tungsten, nickel, and combinations thereof. All or a portion of the filaments may also be composite filaments, such as drawn filled tubes (DFT), as described in US 2016/0249934, which was previously incorporated by reference in its entirety for all purposes. The DFT may include a highly radiopaque core inside a tubing. For instance, the DFT may include a platinum core inside of a nitinol tubing.

Each of the filaments of the plurality of filaments making up the permeable shell 40 have a proximal and distal end. The proximal ends of the filaments of the permeable shell 40 may be gathered in a proximal hub or marker band 68. The device 10 may be attached to a delivery pusher 61 that is disposed within a catheter 70 by attaching the proximal hub or marker band 68 to the distal end of the pusher 61. The distal ends of the plurality of filaments forming the permeable shell 40 may be gathered in a distal marker band 66. Alternatively, the distal ends of the plurality of filaments forming the permeable shell 40 may also be gathered in the proximal hub or marker band 68.

The pusher 61 may be releasably coupled to the device 10. The release of the device 10 from the pusher 61 may be activated by a thermal mechanism, an electrolytic mechanism, a hydraulic mechanism, a shape memory material mechanism, or any other mechanism known in the art of endovascular implant deployment. Embodiments for deployment and release of the device 10 may include connecting the device 10 via a releasable connection to a distal portion of the pusher 61 or other delivery apparatus member. The implant device 10 may be detachably mounted to the distal portion of the pusher 61 by a filamentary tether, string, thread, wire, suture, fiber, or the like, any of which may serve as the tether. The tether may be in the form of a monofilament, rod, ribbon, hollow tube, or the like. Additional details and other methods of attaching the device 10 to the delivery apparatus are described in U.S. Pat. No. 10,932,787, which is hereby expressly incorporated by reference in its entirety for all purposes.

During delivery of the device 10, the operator (e.g., physician) may advance the proximal end of the pusher 61 in a distal direction, thereby advancing the device 10 in a distal direction. If the operator stops advancing the proximal end of the pusher 61, then the device 10 also stops advancing through the catheter 70. However, this may change as the device exits the distal end of the catheter 70, and the self-expanding device 10 can “pull” the pusher in a distal direction, also referred to as “jumping” forward. If this occurs (or is about to occur) and the operator wishes to slow down or stop advancing the device 10 in a distal direction, the operator must apply a proximal force on the pusher 61 to oppose the self-expanding forces of the device 10 pulling it distally and causing the device 10 to jump forward.

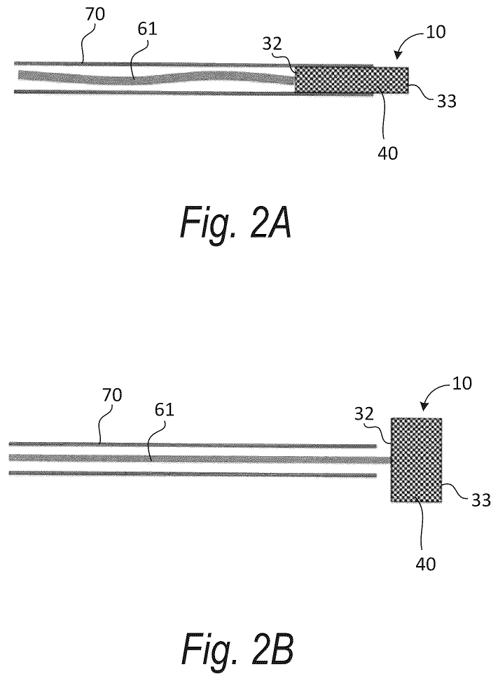

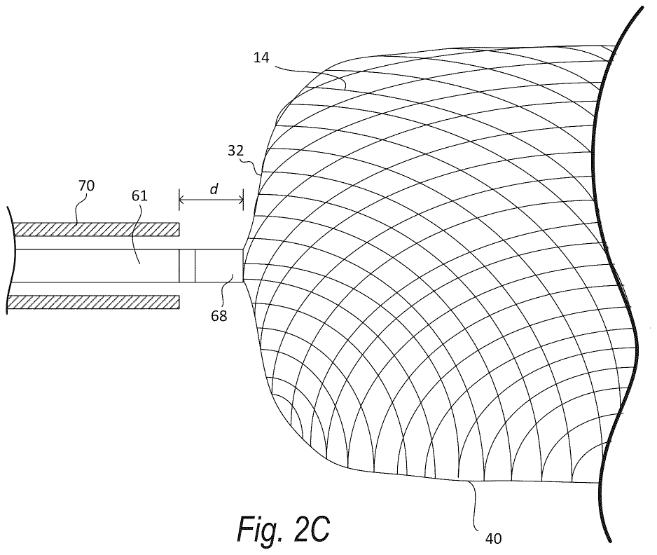

As seen in FIG. 2A, as the distal end 33 of the device 10 is starting to exit the distal end of the catheter 70, the pusher 61 may be slightly buckled due to the friction caused by the permeable shell 40 contacting the inner layer of the catheter 70. As seen in FIGS. 2B and 2C, after the proximal end 32 of the device 10 exits the distal end of the catheter 70, the permeable shell 40 may jump a distance din a direction distal of the distal end of the catheter 70, possibly due to release of the strain on the pusher when there is no more friction between the permeable shell 40 and the inner layer of the catheter 70 after the proximal end 32 of the permeable shell has exited the catheter 70. This sudden movement of implant may be considered undesirable by the physician as exact placement within the vascular defect is very important. For example, a dome of an aneurysm may be very fragile and a sudden, unexpected, or uncontrolled distal movement of the implant during delivery may rupture the aneurysm.

### Delivery System With a Brake

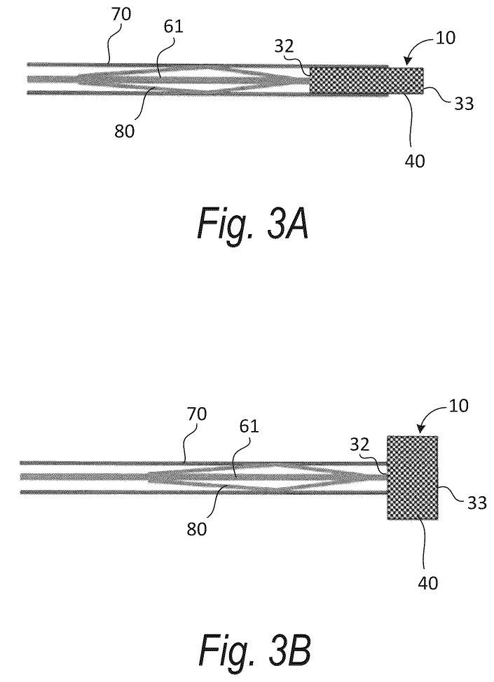

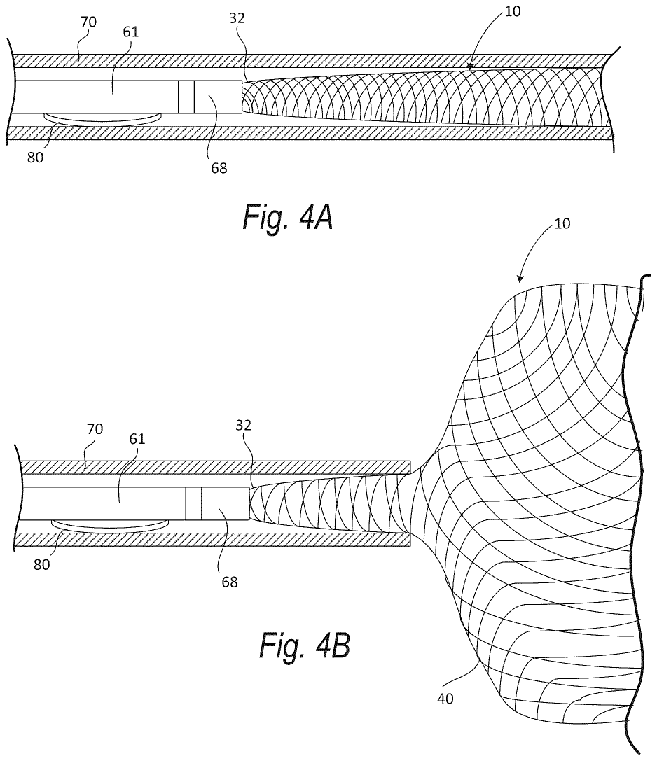

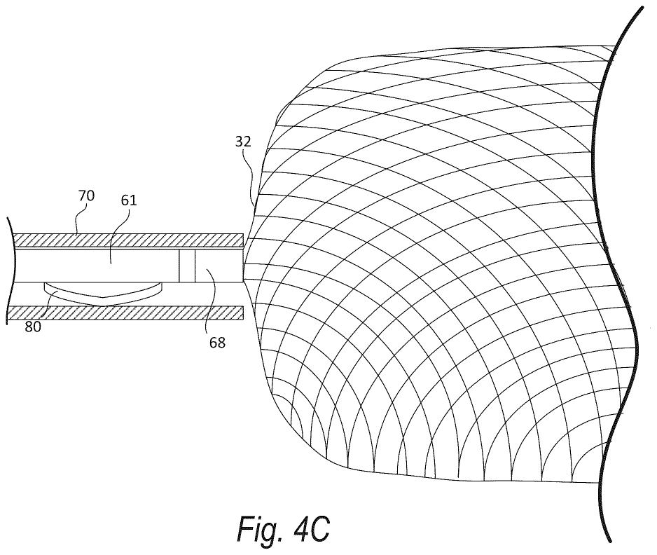

As seen in FIGS. 3A-3B and 4A-4C, a delivery system may include a brake 80 that is coupled or attached to the pusher 61. In some embodiments, the brake 80 may be located in a distal region of the pusher 61 close to the proximal hub 68 of the device 10. The brake 80 may be configured to frictionally engage an inner wall of the catheter 70 such that, even after the device 10 has exited the distal end of the catheter 70 and is no longer in contact with the inner wall of the catheter 70, the brake 80 creates enough frictional force to counteract the release of the buckling of the pusher 61, thereby preventing the device 10 from jumping in a distal direction. Including such a brake is contrary to the delivery systems of the prior art, which emphasize lubricous coatings and other ways to decrease friction during delivery of medical devices. Friction is normally undesirable in delivery systems because it increases the force that the operator must apply to the pusher 61 to advance the device 10, and too much force may cause components of the delivery system to compress or buckle. Friction may also increase the difficulty of navigating sharp turns and/or tortious vasculature and anatomies. For example, at a sharp with a high friction system, increased distal (forward) force from the operator on the pusher 61 may simply drive the device 10 into the lumen of the catheter 70, increasing the static friction at that interface and preventing the device 10 from sliding or otherwise navigating to the target site. For these reasons, in the present system, it may be important for the brake to not engage the lumen of the catheter 70 (or at least not significantly engage the lumen) until the device 10 is near the target site.

The brake may have a braking configuration in which a portion of the brake is in contact with the wall defining a lumen of the catheter 70. In some embodiments, the brake may be in the braking configuration all throughout the catheter lumen, such that a portion of the brake is in contact with the wall defining a lumen of the catheter 70 from a proximal portion of the catheter that is adjacent to a proximal end of the catheter 70 to a distal portion of the catheter that is adjacent to a distal end of the catheter 70.

In some embodiments, the brake 80 may be at least one leaf spring, or one or more leaf springs. As seen in FIGS. 3A-B and 4A-C, the leaf spring may be made from an elongate metallic piece having a first end, a second end, and a curved, bent, or arc-shaped section therebetween. The first and second ends may be attached to the pusher 61, such that the curved or bent section contacts a wall defining a lumen of the catheter 70. In some embodiments, an apex portion of the curved or bent section may contact the wall defining the lumen of the catheter 70. The curved or bent section may apply a pressure to or frictionally engage the wall defining the lumen, thereby creating friction between the brake and the catheter 70.

In some embodiments, the delivery system may include a single leaf spring (see, e.g., FIG. 4A-4C). In other embodiments, the delivery system may include two leaf springs (see, e.g., FIGS. 3A-3B). In other embodiments, the delivery system may include three leaf springs, alternatively four leaf springs, alternatively five leaf springs, alternatively six leaf springs, alternatively at least one leaf spring, alternatively at least two leaf springs, alternatively at least three leaf springs, alternatively at least four leaf springs.

The leaf spring may have a braking configuration in which a portion of the leaf spring is in contact with the wall defining a lumen of the catheter 70. In some embodiments, the leaf spring may be in the braking configuration all throughout the catheter lumen, such that a portion of the leaf spring is in contact with the wall defining a lumen of the catheter 70 from a proximal portion of the catheter that is adjacent to a proximal end of the catheter 70 to a distal portion of the catheter that is adjacent to a distal end of the catheter 70.

Alternative implant embodiments with brakes are depicted in FIGS. 5A-5F. Although the implants are depicted with exemplary leaf spring brake systems, it is to be understood that any of the brake systems described herein may be used with any of the implants described herein. FIG. 5A depicts a single layer occlusive device 10 that includes a permeable shell 40, wherein the filaments 14 are gathered in a proximal and distal marker or hub 68, 66. FIG. 5B depicts a double layer occlusive device having an inverted distal end, where the filaments 14 are gathered in a proximal marker or hub 68. Further details of this implant can be found in PCT/US23/20560, filed May 1, 2023, which is hereby expressly incorporated by reference in its entirety for all purposes. FIG. 5C depicts a multi-layer occlusive device. The device may have two or more layers. In some embodiments, at least two of the layers may be made from the same tubular mesh. The device may also include a ridge or corrugation around a perimeter of the device. In some embodiments, the ridge or corrugation may be around about the middle of the device. Further details of this implant can be found in U.S. Publication No. 2022/0257260, which is hereby expressly incorporated by reference in its entirety for all purposes. FIG. 5D depicts a multi-layer occlusive device in which each of the layers have a different braid angle. In some embodiments, the inner layer may comprise fewer filaments having a larger diameter than or about equal to the outer layer. The inner layer 240 may be a structural layer while the outer layer 140 may be a stasis layer. Alternatively, the inner layer 240 may be a stasis layer while the outer layer 140 may be a structural layer. Further details of this implant can be found in U.S. Publication No. 2021/0282789, which is hereby expressly incorporated by reference in its entirety for all purposes. FIG. 5E depicts an occlusive device with a skirt 82 surrounding at least a portion of a proximal region of the implant. The skirt 82 may assist in preventing blood inflow from the implant in the area at the neck orifice of the aneurysm. The skirt 82 may provide further blood flow restriction into the aneurysm neck.

Further details of this implant can be found in U.S. Publication No. 2023/0114169, which is hereby expressly incorporated by reference in its entirety for all purposes. FIG. 5F depicts an occlusive device having a concave or umbrella shape that sits in the proximal portion of the aneurysm. The device may be heat-set in an inverted configuration similar to a “hat” with a recess near the proximal marker band. The deployed configuration may have a proximal end having a substantially flat portion. Further details of this implant can be found in Intl. Publication No. WO 2023/081340, which is hereby expressly incorporated by reference in its entirety for all purposes.

In some embodiments, the brake may have two configurations, a first configuration in which the brake is not in contact with and/or not frictionally engaged with the inner wall (or lumen) of the catheter 70 and a second configuration in which the brake is frictionally engaged with the inner wall of the catheter 70.

The brake may be made from a shape memory material, such as nitinol. The first configuration of the brake set at a first temperature may not contact the inner wall of the catheter. The second configuration of the brake may be set at a second temperature where the brake expands to the second configuration, which is in contact with or frictionally engaged with the inner wall of the catheter 70. The first temperature may be lower than the second temperature.

The second temperature may be between about 90 °F to about 100 °F, alternatively between about 93 °F to about 100 °F, between about 95 °F to about 100 °F. Thus, the brake may be in the first configuration where it is not in contact with the inner catheter wall when the pusher 61 is first inserted into the catheter 70. As the temperature around the pusher 61 increases once it has been in the patient's body, once the second temperature is reached, the brake may assume the second configuration and may be in the second configuration in the region of interest where the device is deployed.

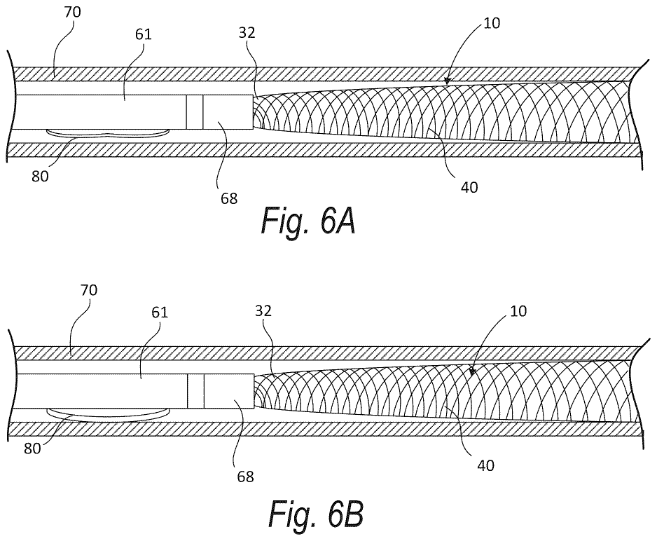

As seen in FIGS. 6A-6B, in one exemplary embodiment where the brake is a leaf spring 80, the first configuration of the leaf spring in FIG. 6A may not be in contact with the inner catheter wall. As the temperature increases past the second temperature, the leaf spring 80 may assume the second, braking configuration, as seen in FIG. 6B, in which the leaf spring 80 is in contact with and/or frictionally engaging the inner wall of the catheter 70. This transition may occur closer to the distal end of the catheter, closer to the region of interest, which would reduce the amount of friction during delivery while the brake could still serve its intended purpose of preventing the implant from jumping forward during deployment of the occlusive device.

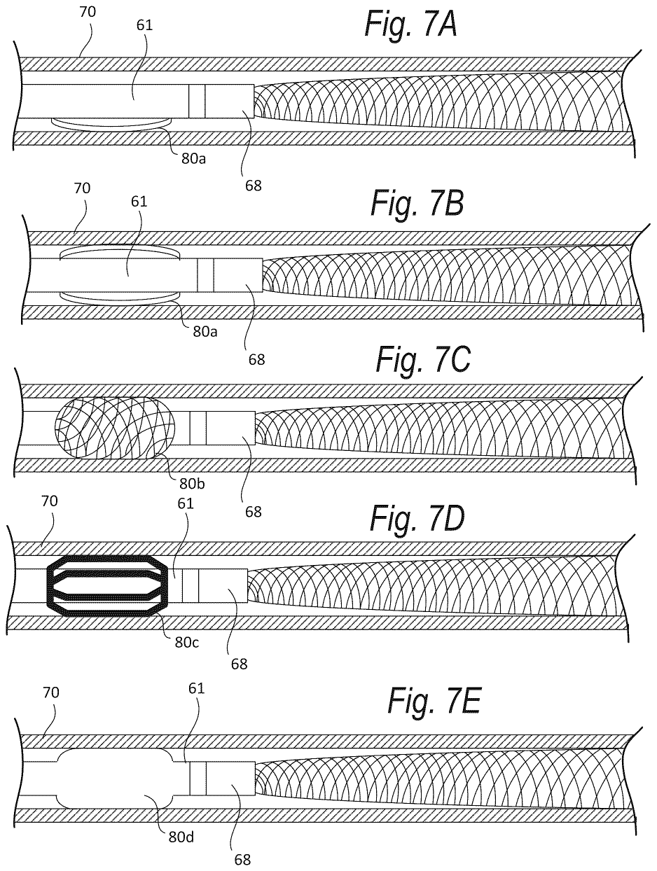

Alternative embodiments of brakes are depicted in FIGS. 7A-7E. FIG. 7A depicts a brake system 80a with leaf spring and FIG. 7B depicts a brake system 80a with two leaf springs. As described previously, each leaf spring may be made from an elongate metallic piece having a first end, a second end, and a curved, bent, or arc-like section therebetween. The first and second ends may be attached to the pusher 61, such that the curved section contacts a wall defining a lumen of the catheter 70. The curved section may apply a pressure to or frictionally engage the wall defining the lumen, thereby creating friction between the brake and the catheter 70. In some embodiments, the leaf springs may be made from a shape memory metal or allow such that it may have different configurations at different temperatures. In such an embodiment, the leaf springs may only be in contact with the inner catheter wall for a portion of the catheter 70 and not the whole length of the catheter 70.

FIG. 7C depicts a braid or tubular mesh braid 80b that may have an outer expanded diameter that is larger than or about equal to an inner diameter of the lumen of the catheter 70. Thus, the braid or tubular mesh 80b may apply a pressure to or frictionally engage the wall defining the lumen, thereby creating friction between the brake and the catheter 70. In some embodiments, the braid or tubular mesh 80b may be made from a shape memory metal or allow such that it may have different configurations at different temperatures. In such an embodiment, the braid or tubular mesh 80b may only be in contact with the inner catheter wall for a portion of the catheter 70 and not the whole length of the catheter 70.

FIG. 7D depicts a laser cut tube 80c having struts 84 that extend outward such that an outer expanded diameter is larger than or about equal to an inner diameter of the lumen of the catheter 70. Thus, the laser cut tube 80c may apply a pressure to or frictionally engage the wall defining the lumen, thereby creating friction between the brake and the catheter 70. In some embodiments, the laser cut tube 80c may be made from a shape memory metal or allow such that it may have different configurations at different temperatures. In such an embodiment, the laser cut tube 80c may only be in contact with the inner catheter wall for a portion of the catheter 70 and not the whole length of the catheter 70.

FIG. 7E depicts a pusher 61 having an area of increased diameter 80d, where the increased diameter is larger than or about equal to an inner diameter of the lumen of the catheter 70. Thus, the area of increased diameter 80d may apply a pressure or frictionally engage to the wall defining the lumen, thereby creating friction between the brake and the catheter 70.

Any of the brake systems described may have first and second configurations, where the first configuration is not in contact with the inner wall of the catheter 70 and wherein the second configuration is in contact with and/or frictionally engaged with the inner wall of the catheter 70. Any of the brake systems described herein may be used with the delivery of any implant described herein. Moreover, any of the brake systems described herein may be used with the delivery of any medical device as it would be understood that such a brake could be applied to pushers for other medical devices such as stent, filters, valves, vascular plugs, flow diverters, and stentrievers/thrombectomy devices.

All features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

As used herein and in the appended claims, the singular forms “a,” “an,” and “the” include plural referents unless the context clearly dictates otherwise.

Aspects of the invention are set out in the independent claims and preferred features are set out in the dependent claims. The preferred features of the dependent claims may be provided in combination in a single embodiment and preferred features of one aspect may be provided in conjunction with other aspects.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

In many embodiments, an implant delivery system includes a self-expandable implant having radially constrained state within a lumen of a catheter and an expanded state after deployment; and a pusher comprising a distal end, a distal region, and a brake located in the distal region of the pusher for frictionally engaging an inner wall of the catheter during deployment of the self-expandable implant, and wherein the implant is releasably coupled to the distal end of the pusher.

In some embodiments, the brake has a resting configuration prior to deployment of the self-expandable implant. In some embodiments, the brake contacts the inner wall of the catheter in the resting configuration. In some embodiments, the brake is not in contact with the inner wall of the catheter in the resting configuration.

In some embodiments, the system further comprises the catheter.

In some embodiments, the brake is at least one leaf spring. In some embodiments, the at least one leaf spring comprises an elongate metallic piece comprising a first end, a second end, and a curved section therebetween, wherein the first and second ends are coupled to the pusher at first and second locations and the curved section contacts the inner wall of the catheter. In some embodiments, the elongate metallic piece comprises nitinol. In some embodiments, the elongate metallic piece comprises stainless steel. In some embodiments, the elongate metallic piece is made from a shape memory metal.

In some embodiments, the brake is at least two leaf springs.

In some embodiments, the brake is made from a shape memory metal, wherein the brake has a first configuration at a first temperature that does not contact the inner wall of the catheter, and a second configuration at a second temperature that does contact the inner wall of the catheter. In some embodiments, the second temperature is between about 90° to about 100°. In some embodiments, the brake is at least one leaf-spring.

In some embodiments, the brake comprises a laser-cut tube.

In some embodiments, the brake comprises a braided mesh.

In some embodiments, the self-expandable implant comprises a plurality of filaments, wherein each of the plurality of filaments has a first end and a second end.

In some embodiments, the plurality of filaments are disposed in a woven structure.

In some embodiments, the first end of each of the plurality of filaments are gathered in a proximal hub at a proximal end of the self-expandable implant.

In some embodiments, the second end of each of the plurality of filaments are gathered in a distal hub at a distal end of the self-expandable implant.

In some embodiments, the second end of each of the plurality of filaments are gathered in the proximal hub at the proximal end of the self-expandable implant.

In some embodiments, the expanded state of the self-expandable implant has a barrel shape.

In some embodiments, the expanded state of the self-expandable implant has an umbrella shape having a concave shape with an open outer end at a distal end of the self-expandable implant.

In many embodiments, an implant delivery system includes a self-expandable implant having radially-constrained state within a lumen of a catheter and an expanded state outside the lumen of the catheter; and a pusher comprising a distal end, a distal region, and a brake means located in the distal region of the pusher for frictionally engaging an inner wall of the catheter to slow a rate that the self-expandable implant expands from the radially-constrained state to the expanded state, wherein the implant is releasably coupled to the distal end of the pusher

In some embodiments, the brake means has a resting configuration prior to deployment of the self-expandable implant. In some embodiments, the brake means contacts the inner wall of the catheter in the resting configuration. In some embodiments, the brake means is not in contact with the inner wall of the catheter in the resting configuration.

In some embodiments, the brake means is at least one leaf spring. In some embodiments, the at least one leaf spring comprises an elongate metallic piece comprising a first end, a second end, and a curved section therebetween, wherein the first and second ends are coupled to the pusher at first and second locations and the curved section contacts the inner wall of the catheter.

In some embodiments, the brake means is made from a shape memory metal, wherein the brake has a first configuration at a first temperature that does not contact the inner wall of the catheter, and a second configuration at a second temperature that does contact the inner wall of the catheter.

In some embodiments, the brake means is made from a shape memory metal, wherein the brake has a first configuration at a first temperature that does not contact the inner wall of the catheter, and a second configuration at a second temperature that does contact the inner wall of the catheter.

In some embodiments, the brake means comprises a laser-cut tube.

In some embodiments, the brake means comprises a braided mesh.

In many embodiments, a method of delivering an implant includes the steps of: advancing, within a catheter, a self-expandable implant coupled to a pusher to a region of interest within a patient's vasculature, wherein the pusher comprises an elongate member having a distal end, a distal region, and a brake associated with the distal region, the self-expandable implant being coupled to the distal end of the pusher and having a radially constrained state at rest within the catheter; deploying the self-expandable implant to an expanded state in the region of interest by advancing the self-expandable implant out of a distal end of the catheter, and where, in a braking configuration during deployment of the self-expandable implant, the brake is frictionally engaged with an inner wall of the catheter while the self-expandable implant expands to the expanded state; and detaching the self-expandable implant from the pusher.

In some embodiments, the brake is at least one leaf spring.

In some embodiments, the at least one leaf spring comprises an elongate metallic piece comprising a first end, a second end, and a curved section therebetween, wherein the first and second ends are coupled to the pusher at first and second locations and the curved section contacts the inner wall of the catheter.

In some embodiments, the elongate metallic piece comprises nitinol.

In some embodiments, the elongate metallic piece comprises stainless steel.

In some embodiments, the elongate metallic piece is made from a shape memory metal.

In some embodiments, the brake is at least two leaf springs.

In some embodiments, the brake has a resting configuration prior to deployment of the self-expandable implant. In some embodiments, the brake contacts the inner wall of the catheter in the resting configuration. In some embodiments, the brake is not in contact with the inner wall of the catheter in the resting configuration.

In some embodiments, the brake is made from a shape memory metal, wherein the brake has a first configuration at a first temperature that does not contact the inner wall of the catheter, and a second configuration at a second temperature that does contact the inner wall of the catheter. In some embodiments, the second temperature is between about 90° to about 100°. In some embodiments, the brake is at least one leaf-spring.

In some embodiments, the brake comprises a laser-cut tube.

In some embodiments, the brake comprises a braided mesh.

In some embodiments, the method further includes the step of withdrawing the catheter from the region of interest.

In some embodiments, the self-expandable implant comprises a plurality of filaments, wherein each of the plurality of filaments has a first end and a second end. In some embodiments, the plurality of filaments are disposed in a woven structure. In some embodiments, the first end of each of the plurality of filaments are gathered in a proximal hub at a proximal end of the self-expandable implant. In some embodiments, the second end of each of the plurality of filaments are gathered in a distal hub at a distal end of the self-expandable implant. In some embodiments, the second end of each of the plurality of filaments are gathered in the proximal hub at the proximal end of the self-expandable implant.

In some embodiments, the expanded state of the self-expandable has a barrel shape.

In some embodiments, the expanded state of the self-expandable implant has an umbrella shape having a concave shape with an open outer end at a distal end of the self-expandable implant.

### Clauses

Exemplary embodiments are set out in the following numbered clauses.

Clause 1. An implant delivery system, comprising:
a self-expandable implant having radially constrained state within a lumen of a catheter and an expanded state after deployment; and
a pusher comprising a distal end, a distal region, and a brake located in the distal region of the pusher for frictionally engaging an inner wall of the catheter during deployment of the self-expandable implant, and wherein the implant is releasably coupled to the distal end of the pusher.

Clause 2. The system of clause 1, wherein the brake has a resting configuration prior to deployment of the self-expandable implant.

Clause 3. The system of clause 2, wherein the brake contacts the inner wall of the catheter in the resting configuration.

Clause 4. The system of clause 2, wherein the brake is not in contact with the inner wall of the catheter in the resting configuration.

Clause 5. The system of clause 1, wherein the system further comprises the catheter.

Clause 6. The system of clause 1, wherein the brake is at least one leaf spring.

Clause 7. The system of clause 1, wherein the brake is at least two leaf springs.

Clause 8. The system of clause 6, wherein the at least one leaf spring comprises an elongate metallic piece comprising a first end, a second end, and a curved section therebetween, wherein the first and second ends are coupled to the pusher at first and second locations and the curved section contacts the inner wall of the catheter.

Clause 9. The system of clause 8, wherein the elongate metallic piece comprises nitinol.

Clause 10. The system of clause 8, wherein the elongate metallic piece comprises stainless steel.

Clause 11. The system of clause 8, wherein the elongate metallic piece is made from a shape memory metal.

Clause 12. The system of clause 1, wherein the brake is made from a shape memory metal, wherein the brake has a first configuration at a first temperature that does not contact the inner wall of the catheter, and a second configuration at a second temperature that does contact the inner wall of the catheter.

Clause 13. The system of clause 12, wherein the second temperature is between about 90° to about 100°.

Clause 14. The system of clause 12, wherein the brake is at least one leaf-spring.

Clause 15. The system of clause 1, wherein the brake comprises a laser-cut tube.

Clause 16. The system of clause 1, wherein the brake comprises a braided mesh.

Clause 17. The system of clause 1, wherein the self-expandable implant comprises a plurality of filaments, wherein each of the plurality of filaments has a first end and a second end.

Clause 18. The system of clause 1, wherein the plurality of filaments are disposed in a woven structure.

Clause 19. The system of clause 1, wherein the first end of each of the plurality of filaments are gathered in a proximal hub at a proximal end of the self-expandable implant.

Clause 20. The system of clause 19, wherein the second end of each of the plurality of filaments are gathered in a distal hub at a distal end of the self-expandable implant.

Clause 21. The system of clause 19, wherein the second end of each of the plurality of filaments are gathered in the proximal hub at the proximal end of the self-expandable implant.

Clause 22. The system of clause 1, wherein the expanded state of the self-expandable implant has a barrel shape.

Clause 23. The system of clause 1, wherein the expanded state of the self-expandable implant has an umbrella shape having a concave shape with an open outer end at a distal end of the self-expandable implant.

Clause 24. A method of delivering an implant, comprising the steps of: advancing, within a catheter, a self-expandable implant coupled to a pusher to a region of interest within a patient's vasculature, wherein the pusher comprises an elongate member having a distal end, a distal region, and a brake associated with the distal region, the self-expandable implant being coupled to the distal end of the pusher and having a radially constrained state at rest within the catheter;
deploying the self-expandable implant to an expanded state in the region of interest by advancing the self-expandable implant out of a distal end of the catheter, and where, in a braking configuration during deployment of the self-expandable implant, the brake is frictionally engaged with an inner wall of the catheter while the self-expandable implant expands to the expanded state; and
detaching the self-expandable implant from the pusher.

Clause 25. The method of clause 24, wherein the brake is at least one leaf spring.

Clause 26. The method of clause 24, wherein the brake is at least two leaf springs.

Clause 27. The method of clause 25, wherein the at least one leaf spring comprises an elongate metallic piece comprising a first end, a second end, and a curved section therebetween, wherein the first and second ends are coupled to the pusher at first and second locations and the curved section contacts the inner wall of the catheter.

Clause 28. The method of clause 27, wherein the elongate metallic piece comprises nitinol.

Clause 29. The method of clause 27, wherein the elongate metallic piece comprises stainless steel.

Clause 30. The method of clause 27, wherein the elongate metallic piece is made from a shape memory metal.

Clause 31. The method of clause 24, wherein the brake has a resting configuration prior to deployment of the self-expandable implant.

Clause 32. The method of clause 31, wherein the brake contacts the inner wall of the catheter in the resting configuration.

Clause 33. The method of clause 31, wherein the brake is not in contact with the inner wall of the catheter in the resting configuration.

Clause 34. The method of clause 24, wherein the brake is made from a shape memory metal, wherein the brake has a first configuration at a first temperature that does not contact the inner wall of the catheter, and a second configuration at a second temperature that does contact the inner wall of the catheter.

Clause 35. The method of clause 34, wherein the second temperature is between about 90° to about 100°.

Clause 36. The method of clause 34, wherein the brake is at least one leaf-spring.

Clause 37. The method of clause 24, wherein the brake comprises a laser-cut tube.

Clause 38. The method of clause 24, wherein the brake comprises a braided mesh.

Clause 39. The method of clause 24, further comprising the step of withdrawing the catheter from the region of interest.

Clause 40. The method of clause 24, wherein the self-expandable implant comprises a plurality of filaments, wherein each of the plurality of filaments has a first end and a second end.

Clause 41. The method of clause 40, wherein the plurality of filaments are disposed in a woven structure.

Clause 42. The method of clause 40, wherein the first end of each of the plurality of filaments are gathered in a proximal hub at a proximal end of the self-expandable implant.

Clause 43. The method of clause 42, wherein the second end of each of the plurality of filaments are gathered in a distal hub at a distal end of the self-expandable implant.

Clause 44. The method of clause 42, wherein the second end of each of the plurality of filaments are gathered in the proximal hub at the proximal end of the self-expandable implant.

Clause 45. The method of clause 40, wherein the expanded state of the self-expandable has a barrel shape.

Clause 46. The method of clause 40, wherein the expanded state of the self-expandable implant has an umbrella shape having a concave shape with an open outer end at a distal end of the self-expandable implant.

Clause 47. An implant delivery system, comprising: a self-expandable implant having radially-constrained state within a lumen of a catheter and an expanded state outside the lumen of the catheter; and a pusher comprising a distal end, a distal region, and a brake means located in the distal region of the pusher for frictionally engaging an inner wall of the catheter to slow a rate that the self-expandable implant expands from the radially-constrained state to the expanded state, wherein the implant is releasably coupled to the distal end of the pusher.

Clause 48. The system of clause 47, wherein the brake means has a resting configuration prior to deployment of the self-expandable implant.

Clause 49. The system of clause 48, wherein the brake means contacts the inner wall of the catheter in the resting configuration.

Clause 50. The system of clause 48, wherein the brake means is not in contact with the inner wall of the catheter in the resting configuration.

Clause 51. The system of clause 47, wherein the brake means is at least one leaf spring.

Clause 52. The system of clause 51, wherein the at least one leaf spring comprises an elongate metallic piece comprising a first end, a second end, and a curved section therebetween, wherein the first and second ends are coupled to the pusher at first and second locations and the curved section contacts the inner wall of the catheter.

Clause 53. The system of clause 47, wherein the brake means is made from a shape memory metal, wherein the brake has a first configuration at a first temperature that does not contact the inner wall of the catheter, and a second configuration at a second temperature that does contact the inner wall of the catheter.

Clause 54. The system of clause 47, wherein the brake means is made from a shape memory metal, wherein the brake has a first configuration at a first temperature that does not contact the inner wall of the catheter, and a second configuration at a second temperature that does contact the inner wall of the catheter.

Clause 55. The system of clause 47, wherein the brake means comprises a laser-cut tube.

Clause 56. The system of clause 47, wherein the brake means comprises a braided mesh.

1. An implant delivery system, comprising:
a self-expandable implant having radially constrained state within a lumen of a catheter and an expanded state after deployment; and
a pusher comprising a distal end, a distal region, and a brake located in the distal region of the pusher for frictionally engaging an inner wall of the catheter during deployment of the self-expandable implant, and wherein the implant is releasably coupled to the distal end of the pusher.

2. The system of claim 1, wherein the brake has a resting configuration prior to deployment of the self-expandable implant.

3. The system of claim 2, wherein the brake contacts the inner wall of the catheter in the resting configuration.

4. The system of claim 2, wherein the brake is not in contact with the inner wall of the catheter in the resting configuration.

5. The system of claim 1, wherein the system further comprises the catheter.

6. The system of claim 1, wherein the brake is at least one leaf spring.

7. The system of claim 1, wherein the brake is at least two leaf springs.

8. The system of claim 6, wherein the at least one leaf spring comprises an elongate metallic piece comprising a first end, a second end, and a curved section therebetween, wherein the first and second ends are coupled to the pusher at first and second locations and the curved section contacts the inner wall of the catheter.

9. The system of claim 8, wherein the elongate metallic piece comprises nitinol.

10. The system of claim 8, wherein the elongate metallic piece comprises stainless steel.

11. The system of claim 8, wherein the elongate metallic piece is made from a shape memory metal.

12. The system of claim 1, wherein the brake is made from a shape memory metal, wherein the brake has a first configuration at a first temperature that does not contact the inner wall of the catheter, and a second configuration at a second temperature that does contact the inner wall of the catheter.

13. The system of claim 12, wherein the second temperature is between about 90° to about 100°.

14. The system of claim 12, wherein the brake is at least one leaf-spring.

15. The system of claim 1, wherein the brake comprises a laser-cut tube.

16. The system of claim 1, wherein the brake comprises a braided mesh.

17. The system of claim 1, wherein the self-expandable implant comprises a plurality of filaments, wherein each of the plurality of filaments has a first end and a second end.

18. (canceled)

19. The system of claim 1, wherein the first end of each of the plurality of filaments are gathered in a proximal hub at a proximal end of the self-expandable implant.

20. The system of claim 19, wherein the second end of each of the plurality of filaments are gathered in a distal hub at a distal end of the self-expandable implant.

21. The system of claim 19, wherein the second end of each of the plurality of filaments are gathered in the proximal hub at the proximal end of the self-expandable implant.

22-33. (canceled)
